(19) United States
(12) Patent Application Publication
(10) Pub. No.: US 2017/0066728 A1
(43) Pub. Date: Mar. 9, 2017

(54) PROCESS FOR THE PREPARATION OF STABLE CRYSTALLINE FORM-I OF LINEZOLID, SUBSTANTIALLY FREE OF RESIDUAL SOLVENT

(71) Applicant: Jubilant Life Sciences Limited, Noida (IN)

(72) Inventors: Sujay BISWAS, Noida (IN); Atulya Kumar PANDA, Noida (IN); Ashish Kumar GUPTA, Noida (IN); Shishupal SINGH, Noida (IN); Praveen TIWARI, Noida (IN); Dharam VIR, Noida (IN); Saji THOMAS, Noida (IN)

(21) Appl. No.: 15/356,374

(22) Filed: Nov. 18, 2016

Related U.S. Application Data
(62) Division of application No. 14/374,213, filed on Jul. 23, 2014.

(30) Foreign Application Priority Data
Jan. 24, 2012 (IN) 200/DEL/2012
Jan. 31, 2012 (IN) 267/DEL/2012
Jul. 19, 2012 (IN) 2254/DEL/2012

(51) Int. Cl. C07D 263/24

(52) U.S. Cl. CPC C07D 263/24; C07B 2200/07; C07B 2200/13

(57) ABSTRACT

The invention relates to a process of preparation of enantiomerically pure Linezolid Form-I comprising converting a substantially enantiomerically pure linezolid hydroxide compound of formula II to Linezolid Form I compound of formula I.

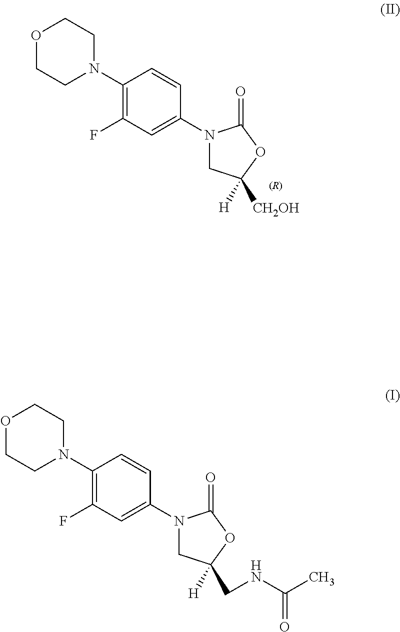

### FIELD OF THE INVENTION

The present invention relates to an improved processes for the enantiomerically pure linezolid compound of formula-I. In particular, the present invention is directed to a novel process for enantiomerically pure linezolid hydroxide compound of formula-II, which provides enantiomeric purity more than 99.9% of R-isomer relative to its S-isomer. In the further aspect of present invention also provides conversion linezolid hydroxide to linezolid, having S-isomer content more than 99.9% relative to R-isomer. Moreover, the present invention relates to an substantially enantiomerically pure R-isomer linezolid hydroxide compound of formula-II in a very high degree of enantiomeric purity as relative to its S-isomer and its use in subsequent conversion into linezolid compound of formula-I.

The present invention also relates to the to a enantiomeric pure linezolid Form-I having S-isomer content more than about 99.9% relative to its R-isomer. Further aspect of invention provides improved processes for preparation of enantiomeric pure linezolid Form-I of formula-I. In particular, the present invention is directed to a novel process for preparation of enantiomeric pure linezolid Form-I of formula-I, which provides enantiomeric pure linezolid Form-I, having the polymorphic purity more than 99.9% as relative to any other known polymorphic form of linezolid. In the further aspect of present invention also provides conversion enantiomeric pure linezolid Form-I of formula-I to any other form of linezolid.

The present invention also relates to stable linezolid form I, which is substantially free of residual solvent(s). The present invention also relates to improved process for the preparation of polymorphic form I of enantiomerically pure linezolid compound of formula-I substantially free of residual solvent(s). In particular, the present invention is directed to process for the preparation of stable crystalline form I of linezolid, substantially free of solvent(s) by applying the reaction procedure as described herein or subjecting linezolid having residual solvent(s) to de-solvent treatment, in an industrially advantageous method.

### BACKGROUND OF THE INVENTION

Linezolid, (S)—N-[[3-(3-fluoro-4-morpholinylphenyl)-2-oxo-5-oxazolidinyl]methyl] acetamide compound of formula-I is an antimicrobial agent. Linezolid is an oxazolidinone, having the empirical formula C₁₆H₂₀FN₃O₄

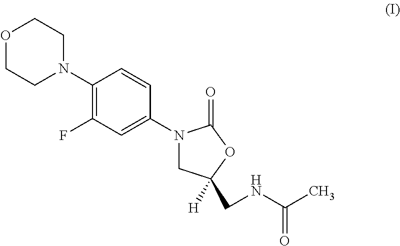 [SMILES: CC(=O)NC[C@H]1CN(c2ccc(N3CCOCC3)c(F)c2)C(=O)O1]

U.S. Pat. No. 5,688,792 describe the linezolid and its use for the treatment of microbial infections. The processes for their preparation of linezolid were described in several patents and patent applications including U.S. Pat. No. 5,688,792, U.S. Pat. No. 5,837,870, U.S. Pat. No. 7,291,614, WO 99/24393, as well as the journal articles such as J. Med. Chem. 39(3), 673-679, 1996 and Tetrahedron Lett 40(26), 4855, 1999.

Linezolid is known to exhibit polymorphism. U.S. Pat. No. 6,559,305 and U.S. Pat. No. 6,444,813 addressed that the product obtained by the process described in product patent of linezolid i.e. U.S. Pat. No. 5,688,792 (the '792 patent) and J. Med. Chem. 39(3), 673-679, 1996 are polymorphic Form-I. The '792 patent described process involves the use of silica gel column with eluting a gradient of 2-10% methanol/ethyl acetate (v/v).

J. Med. Chem. 39(3), 673-679, 1996 described process, which involves the use of ethyl acetate and hexane to recrystallize the linezolid.

The International Patent Application WO 2011077310 describes the process for preparation linezolid Form-I, which involves final recrystallization from the ketonic solvents.

The key constraint in the prior art process is to achieve the pharmaceutically acceptable enantiomerically pure linezolid. The rational of the drawback is lack of enantiomeric purity in the advanced intermediates of linezolid such as linezolid hydroxide of formula-II.

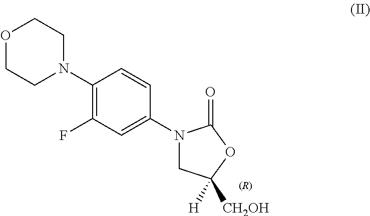

Hence, there is a need to have enantiomerically pure intermediate to prepare enantiomerically pure linezolid of formula-I.

[(R)—N-[[3-(3-Fluoro-4-morpholinyl)phenyl]-2-oxo-5-oxazolidinyl]methanol] i.e. linezolid hydroxide of formula-II is an intermediate used in the synthesis of linezolid. Linezolid hydroxide is an advanced key intermediate for the synthesis of linezolid.

There are a number of methods for preparing linezolid hydroxide described in the prior art. U.S. Pat. No. 5,688,792 describes crystallization from a mixture of ethyl acetate and hexane.

International Patent Application No. WO 2009/032294 ('294 application) describes substantially pure linezolid hydroxide and its purification from the solvent selected from the alcohol and ketone solvents. The '294 application discloses the significance of enantiomeric purity of linezolid hydroxide, which is used as an advanced key intermediate for the process of preparation of linezolid of formula-I.

International Patent Application No. WO 2010/084514 describes the process of purification linezolid hydroxide from the ethyl acetate and water. However the purity of linezolid hydroxide obtained by all said prior art are from 98% to 99.8%, which reflects the enantiomeric purity of linezolid active pharmaceutical ingredient itself. Thus, therefore remains a need to obtain highly pure linezolid hydroxide and its subsequent conversion to pure linezolid.

The above mentioned processes are also not able to provide pure polymorphic form I with enantiomerically pure linezolid of desired pharmaceutical purity on industrial scale. Furthermore, product patent, i.e. U.S. Pat. No. 5,688,792 describes a process that needs column chromatography for the purification of final compound, which is cumbersome technique and difficult to practice during commercial-scale production and practice of such techniques requires large quantities of solvent and its subsequent recovery, which ultimately increases the overall cost of production of pharmaceutically acceptable linezolid.

U.S. Pat. No. 5,837,870 describes crystallization of linezolid hydroxide from mixture of ethyl acetate, heptane and water. U.S. Pat. No. 5,837,870 also discloses a method of crystallization of linezolid hydroxide by means of dissolving linezolid in hot ethyl acetate and addition of heptane.

U.S. Pat. No. 6,559,305 discloses and claims linezolid crystalline Form II, which is characterized by its XRD and IR values.

The International Patent Application WO 2011077310 describes the process for preparation linezolid Form-I, which involves final recrystallization from the ketonic solvents. Pharmaceutical Research, (2008), 25, 530, explains that the ability to deliver the drug to the patient in a safe, efficacious and cost effective way depends largely upon the physicochemical properties of the APIs in the solid state and accordingly one of the challenging tasks in the pharmaceutical industry is to design pharmaceutical materials with specific physiochemical properties. It is known that different solid forms of the same drug may exhibit different properties, including characteristics that have functional implications with respect to their use as drug may have substantial differences in such pharmaceutically important properties as dissolution rates and bioavailability. Likewise, different polymorphs may have different processing properties, such as hygroscopicity, flow ability and the like, which could affect their suitability as active pharmaceuticals for commercial production. Also, it is known in the art that the amorphous forms of APIs generally exhibit the better solubility profile over the corresponding crystalline forms. This is because the lattice energy does not have to be overcome in order to dissolve the solid state structure as in the case for crystalline forms.

None of the above mentioned prior arts offer simple and cost effective process for the preparation of enantiomerically pure linezolid having stable Form-I of formula-I. Thus, there is a need to develop the solid state forms of pharmaceutically active compound, in substantially pure form having better physicochemical properties; especially, for the enhancement of the solubility. Also there is a constant need to have the cost effective and industrial friendly process for the preparation of the solid state form in substantially pure linezolid having stable Form-I of formula-I, which is reproducible.

### SUMMARY OF THE INVENTION

The present invention seeks to overcome the prior art limitations and to provide a cost effective and industrially favorable advanced intermediate of linezolid formula I, in the form of substantially pure linezolid hydroxide formula II, wherein the linezolid hydroxide compound having a R-isomer content is more than about 99.9% relative to its S-isomer, while avoiding cumbersome purification process such as chromatography or repeated crystallization.

The present invention also encompasses a process for the enantiomeric pure linezolid hydroxide compound of formula-II.

The present invention also encompasses substantially enantiomerically pure linezolid hydroxide compound of formula-II, which is subsequently converted into linezolid formula-I.

The present invention seeks to overcome the prior art limitations and to provide a cost-effective and industrially favorable enantiomerically pure linezolid Form-I of formula-I, having S-isomer content more than about 99.9% relative to its R-isomer,

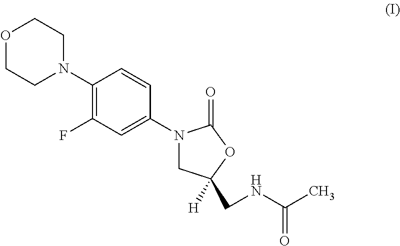 [SMILES: CC(=O)NC[C@H]1CN(c2ccc(N3CCOCC3)c(F)c2)C(=O)O1]

which is characterized by an X-ray powder diffraction spectrum having peaks expressed as 2θ at about 7.3, 9.3, 13.4, 14.7, 15.3, 16.8, 17.9, 18.4, 18.9, 20.9, 21.2, 22.1 and 25.3 degrees.

The present invention also encompasses improved process for preparation of enantiomerically pure Form-I of linezolid of formula-I

The present invention also encompasses enantiomerically pure linezolid Form-I of formula-I, which is subsequently converted into any other known polymorphic form of linezolid.

The present invention also encompasses substantially solvent free stable crystalline Form-I of linezolid. The solvent free stable crystalline Form-I of linezolid as described herein is linezolid having residual solvent(s) less than about 1200 ppm, preferably less than about 1000 ppm.

The present invention also encompasses the process for the preparation of a stable and substantially solvent-free crystal of Form-I of linezolid.

The present invention also encompasses the process for the preparation of more than about 99.9% pure linezolid free from bislinezolid the process comprising removal of N,N-bis[[(5S)-3-[3-fluoro-4-(4-morpholinyl)phenyl]-2-oxo-5-oxazolidinyl]methyl]amine (amino dimer impurity) compound of formula III from the reaction mixture of (S)-[[N-3-(3-Fluoro-4-morpholinylphenyl)-2-oxo-5-oxazolidinyl]methyl] amine p-TSA salt in hydrochloric acid at pH to 4.5-4.7 by washing the solution with ester solvent.

The present invention also encompasses the process for the preparation of more than 99.9% pure linezolid free N-(2-{[3-fluoro-4-(morpholine-4-yl) phenyl]amino}-1-hydroxyethyl)acetamide compound of formula IV from the reaction mixture of (S)-[[N-3-(3-Fluoro-4-morpholinylphenyl)-2-oxo-5-oxazolidinyl]methyl] amine p-TSA salt, acetic anhydride and water at pH to 7-7.5 by extraction using chlorinated solvent.

### DETAILED DESCRIPTION OF THE INVENTION

For purposes of the present invention, “substantially enantiomerically pure” means linezolid hydroxide having enantiomeric purity more than 99.9% of R-isomer relative to its S-isomer. Preferably, the R-isomer linezolid hydroxide having more than 99.93% and more preferably more than 99.95%, as measured by HPLC methods.

For purposes of the present invention, “substantially enantiomerically pure” means linezolid having enantiomeric purity more than 99.9% of S-isomer relative to its R-isomer, preferably, the S-isomer linezolid having more than 99.93% and more preferably more than 99.95%, as measured by HPLC methods.

For purposes of the present invention, “enantiomeric pure or enatiomerically pure” means linezolid having enantiomeric purity more than 99.9% of S-isomer relative to its R-isomer. preferably, the S-isomer linezolid having more than 99.93% and more preferably more than 99.95%, as measured by HPLC methods.

The present invention provides a substantially enantiomerically pure linezolid hydroxide formula II.

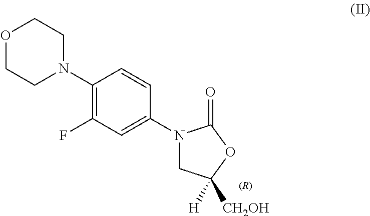

wherein the compound of formula II, having a R-isomer content more than about 99.9% relative to its S-isomer, while avoiding cumbersome purification process such as chromatography or repeated crystallization.

Further, the process of present invention involves the process for enantiomerically pure linezolid hydroxide, wherein linezolid hydroxide is directly isolated from the reaction mixture without isolating any separate purification step.

In one embodiment of the present invention also encompasses a process for the enantiomeric pure linezolid hydroxide compound of formula-II. The process for enantiomeric pure linezolid hydroxide comprises the steps of

(a) contacting linezolid hydroxide compound of formula-II and an ester solvent.

(b) optionally adjusting the moisture content of the solution of step (a) in between 0.2 to 0.6% w/w.

(c) optionally adding anti solvent.

(d) isolating linezolid hydroxide.

One another embodiment of the present invention relates to conversion of substantially pure linezolid hydroxide to linezolid by any means known in the art. Linezolid produced can be used in the preparation of a medicament.

[(R)—N-[[3-(3-Fluoro-4-morpholinyl)phenyl]-2-oxo-5-oxazolidinyl]methanol] (linezolid hydroxide) is an intermediate used in the synthesis of linezolid. Linezolid hydroxide can be prepared by any method known in the prior art.

The moisture content of solvent may be maintained by means of adding required quantity of water or removing the excess water from the solution.

The moisture content of solution is maintained in between 0.2-0.6% w/w, preferably, 0.25-0.55% w/w.

Linezolid hydroxide obtained from the reaction mixture can be directly used upon removal of solvents. Alternatively, the solution of linezolid hydroxide is prepared by dissolving linezolid hydroxide in the solvent, for example by heating or by stirring for a sufficient period of time to dissolve the linezolid hydroxide.

The ester solvent is selected from the group comprising of methyl acetate, ethyl acetate, n-propyl acetate, isopropyl acetate, n-butyl acetate and mixtures thereof, preferably, the solvent is ethyl acetate.

The antisolvent may be selected form a group comprising of cyclic and non-cyclic linear or branched chain hydrocarbon, preferably, pentane, hexane, heptane, octane, cyclohexane, methylcyclohexane, chloronaphthalene, orthodichlorbenzene, toluene, ethylbenzene, isopropylbenzene, diethylbenzene and mixtures thereof, more preferably, the antisolvent is hexane or cyclohexane or heptane.

Once enantiomerically pure linezolid hydroxide is obtained, it can be isolated by any means known in the art.

In another embodiment present invention includes the repetition of the process for purification of linezolid hydroxide to further increase the content of the R-isomer. The repetition is dependent of the enantiomeric purity of linezolid hydroxide.

The weight to volume ratio [g/mL] of linezolid hydroxide to solvent is preferably from about 1:6 to about 1:12, preferably from about 1:8 to about 1:10.

The invention relates to enantiomerically pure linezolid hydroxide, obtained by process of present invention, having enantiomeric purity more than 99.9% of R-isomer relative to its S-isomer. Preferably, the R-isomer linezolid hydroxide having more than 99.93% and more preferably, more than 99.95%, as measured by HPLC methods.

Linezolid hydroxide may be obtained from the any process in the art or the process described in application WO2011/114210.

The resulting substantially pure linezolid hydroxide can be subsequently converted to linezolid by any means known in the art as well as by the process described in application WO2011/114210. Linezolid produced can then be used in the preparation of a medicament.

The process for preparation of linezolid hydroxide according to the present invention can be carried out by isolating the intermediate or one-pot reaction or without isolating the intermediate compounds, starting from steps: (a) condensation of 3,4-difluoronitrobenzene with morpholine to obtain 3-fluoro-4-morpholinyl nitrobenzene; (b) reduction of obtained compound in step ‘a’ to 3-fluoro-4-morpholinyl aniline; (c) carbamoylation of amino group of obtained compound in step ‘b’ to generate carbamate derivative like ethyl or benzyl carbamate and the like; (d) N-alkylation of obtained ethyl carbamate derivative or benzyl carbamate derivative in step (c) with (R)-glycidyl butyrate followed by in-situ cyclization to obtain (R)—N-[3-(3-fluoro-4-morpholinylphenyl)-2-oxo-5-oxazolidinyl]methanol.

In an another embodiment the present invention provides enantiomerically pure linezolid Form-I of formula-I, having S-isomer content more than about 99.9% relative to its R-isomer.

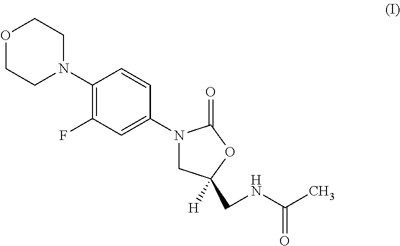 [SMILES: CC(=O)NC[C@H]1CN(c2ccc(N3CCOCC3)c(F)c2)C(=O)O1]

which characterized by an X-ray powder diffraction spectrum having peaks expressed as 2θ at about 7.3, 9.3, 13.4, 14.7, 15.3, 16.8, 17.9, 18.4, 18.9, 20.9, 21.2, 22.1 and 25.3 degrees.

For purposes of the present invention, enantiomerically pure linezolid Form-I is characterized by an x-ray powder diffraction spectrum substantially similar to the XRD in FIG. 1

For purposes of the present invention, “the purity of linezolid Form-I” is more than 99.0% relative other polymorphic form of linezolid. Preferably, “the purity of linezolid Form-I” is more than 99.5% relative other polymorphic form of linezolid, more preferably “the purity of linezolid Form-I” more than 99.9%.

In one embodiment of present invention also encompasses improved process for preparing enantiomerically pure linezolid Form-I of linezolid of formula-I comprises the step of:
(a) providing a solution or slurry or suspension of linezolid in a solvent at the suitable temperature,
(b) mixing same solvent or optionally antisolvent with the solution or slurry or suspension as obtained from step (a) at temperature lower than temperature in step (a) and
(c) isolation of enantiomerically pure Form-I of linezolid.

The suitable temperature of step (a) is about 30° C. to about 150° C., preferably, about 45° C. to about 60° C.

The temperature of step (b) is about 30° C. to about −30° C., preferably, about 10° C. to about −20° C.

Further, the process of present invention involves the process for the preparation of enantiomeric pure linezolid Form-I, wherein linezolid Form-I is directly isolated from the reaction mixture without involving any separate purification step.

One another embodiment of the present invention relates to conversion of enantiomerically pure linezolid Form-I to any other known polymorphic form of linezolid.

Linezolid, (S)—N-[[3-(3-fluoro-4-morpholinylphenyl)-2-oxo-5-oxazolidinyl]methyl] acetamide of formula-I, can be prepared by any method known in the prior art.

Linezolid obtained from the reaction mixture can be directly used upon removal of solvents. Alternatively, the solution of linezolid is prepared by dissolving linezolid in the solvent, for example by heating or by stirring for a sufficient period of time to dissolve the linezolid.

The solvent are ester solvents, halogenated solvents, ketonic solvents, and ethers solvents.

The ester solvent is selected from the group comprising of methyl acetate, ethyl acetate, n-propyl acetate, isopropyl acetate, n-butyl acetate and mixtures thereof and the like, preferably, the solvent is ethyl acetate.

The halogenated solvent is selected from the group comprising of dichloromethane (DCM), chloroform, dichloroethane, chlorobenzene, chloroform, mixtures thereof and the like.

The ketonic solvent is selected from the group comprising of acetone, methyl isobutyl ketone (MIBK), methyl isopropyl ketone (MIPK) and methyl ethyl ketone (MEK), mixtures thereof and the like.

The ether solvent is selected from the group comprising of tetrahydrofuran (THF), dioxane, methyl tert. butyl ether, mixtures thereof and the like.

Antisolvent is hydrocarbon solvent, wherein hydrocarbon solvent is selected from the group comprising of n-hexane, n-heptane, cyclohexane, toluene, xylenes and mixtures thereof. Preferably, the antisolvent is n-hexane, n-heptane, and cyclohexane, mixtures thereof and the like.

Once enantiomerically pure linezolid Form-I of formula-I is obtained, it can be isolated by any means known in the art.

The resulting enantiomeric pure linezolid having enantiomeric purity more than 99.9% of S-isomer relative to its R-isomer. Preferably, the S-isomer linezolid having more than 99.93% and more preferably more than 99.95%, as measured by HPLC methods.

Linezolid may be obtained from the any process in the art or the process described in PCT application WO2011/114210.

The process for preparation of linezolid according to the present invention can be carried out by isolating the intermediate or one-pot reaction or without isolating the intermediate compounds, starting from steps: (a) condensation of 3,4-difluoronitrobenzene with morpholine to obtain 3-fluoro-4-morpholinyl nitrobenzene; (b) reduction of obtained compound in step ‘a’ to 3-fluoro-4-morpholinyl aniline; (c) carbamoylation of amino group of obtained compound in step ‘b’ to generate carbamate derivative like ethyl or benzyl carbamate and the like; (d) N-alkylation of obtained ethyl carbamate derivative or benzyl carbamate derivative in step (c) with (R)-glycidyl butyrate followed by in-situ cyclization and hydrolysis to obtain (R)—N-[3-(3-fluoro-4-morpholinylphenyl)-2-oxo-5-oxazolidinyl]methanol. (e) further, conversion of the hydroxy group as obtained in step ‘d’ into a leaving group e.g. mesylate, nosylate, tosylate, triflate, besylate or a halo compound. If the leaving group is tosylate the compound generated is (R)—N-[3-(3-fluoro-4-morpholinylphenyl)-2-oxo-5-oxazolidinyl]methyl p-toluenesulfonate; (f) conversion of tosylate compound as obtained in step ‘e’ to (S)—N-[[3-(3-fluoro-4-morpholinylphenyl)-2-oxo-5-oxazolidinyl]methyl]amine p-TSA salt, and (g) acetylation of obtained p-TSA salt compound as obtained in step ‘f’ to provide linezolid of formula-I.

It has been observed by the inventors of the present invention that linezolid Form I containing high level of residual solvent triggers the formation of polymorphic impurity, Form-II of linezolid. Therefore, it is most critical to have a control on residual solvent to manufacture stable and pure Form-I of linezolid, irrespective of the polymorphic purity we may achieve during synthesis. Form I of linezolid tends to lose polymorphic stability and undergoes transformation into Form II when it contains residual solvent(s), though complying with the regulatory norms. Therefore, residual solvent in the crystal must be reduced to minimum level. We have observed that once the material i.e. Form-I of linezolid is substantially free of solvent(s), it does not generate polymorphic impurity, Form-II. Thus, it remains as the stable Form I.

However, when the production procedure for linezolid as described in the above mentioned prior arts is followed, solvent(s) can hardly be eliminated from the product and the resulting solid inevitably contains fair amount of solvent(s). Thus, linezolid provided by the processes described in the above literature contains residual solvent(s) and it is difficult to desolvate from the crystal i.e. to reduce the content of residual solvent(s) from the compound by without detracting from the stability of the product.

We have observed that linezolid having more than 1000 ppm of residual solvents reduces the purity of the product. Therefore, there has been a demand for a process for producing substantially solvent-free crystals of Form-I of linezolid, which can be developed on production-scale in an industrial friendly manner. Therefore, to achieve residual solvent content less than 1000 ppm in Form-I of linezolid is the most critical quality attribute for the polymorphic purity.

Substantially solvent free crystals of Form-I of linezolid as described herein is linezolid having residual solvent(s) less than about 1200 ppm, preferably less than about 1000 ppm.

In view of the above state of the art, we did an intensive investigation directed to improvements in the above-mentioned aspects for the purpose of providing stable and substantially solvent-free crystals of Form-I of linezolid, which is of value as a medicine, for example as an antimicrobial agent and so forth for the development of industrial friendly technology for providing such crystals.

Therefore, in an embodiment the invention provides stable substantially solvent-free crystal of linezolid.

In an embodiment the invention provides stable substantially solvent-free crystal of linezolid Form I.

In an embodiment the invention provides substantially solvent-free crystal of linezolid, wherein the solvent content of the substantially solvent free crystal(s) is less than about 1200 ppm preferably less than about 1000 ppm.

In still another embodiment, the invention provides a process for the preparation of a stable and substantially solvent-free crystal of Form-I of linezolid, which can be achieved by a process comprising the steps of:
(a) providing a solution of linezolid in a solvent at a first temperature;
(b) addition of the solution obtained in step (a) into a pre-cooled solvent at a second temperature;
(c) stirring the solution of step (b) at a temperature which is not more than about 5° C.; (d) optionally repeating the steps (b) and (c);
(e) isolation of substantially solvent-free crystals of Form-I of linezolid and
(f) drying the material obtained in step (e) at a temperature above about 90° C.

The first temperature in step (a) at which linezolid is dissolved in a solvent system is a temperature range between about 50° C. and refluxing temperature of the solvent system; preferably between about 55° C. and refluxing temperature of the solvent system. The pre-cooled solvent mentioned in step (b) is a temperature of the solvent ranging from about −10° C. to about −5° C.; and the second temperature mentioned in step (b) is a temperature ranging from about −10° C. or −5° C. to about 20° C.; preferably about −10° C. to about 15° C.

The temperature of above about 90° C. as mentioned in step (f) is a temperature ranging from about 90° C. to about 140° C., preferably from about 100° C. to about 120° C., more preferably from about 100° C. to about 110° C.; the drying performed in step (f) is preferably under vacuum.

The solvent used in steps (a) and (b) is selected from the group comprising of esters, alcohols, nitriles, ketones, ethers, amides, dialkylsulfoxide, chlorinated solvents or the mixtures thereof. Esters are selected from the group comprising of ethyl acetate, propyl acetate and the like; preferably ethyl acetate. Alcohols are selected from the group comprising of methanol, ethanol, n-propanol, isopropanol, n-butanol and the like. The nitriles are selected from the group comprising of acetonitrile, propionitrile, butyronitrile, valeronitrile and the like. Ketones are selected from the group comprising of acetone, methyl ethyl ketone, methyl isobutyl ketone etc. Chlorinated solvents are selected from the group comprising of dichloromethane, chloroform, dichloroethane, chlorobenzene and the like. Ethers can be selected from the group comprising of diisopropyl ether, tetrahydrofuran, dioxane and the like. Amides can be selected from the group comprising of dimethylformamide, dimethylacetamide, N-methyl formamide and the like. Dialkyl sulfoxide can be selected from the group comprising of dimethyl sulfoxide, diethyl sulfoxide, dibutyl sulfoxide and the like.

In still another embodiment, the invention provides a process for the preparation of a stable and substantially solvent-free crystal of Form-I of linezolid, which can be achieved by a process comprising the steps of:
a) providing a solution of linezolid in an organic solvent or mixture thereof or a mixture of organic solvent and water;
b) removal of solvent using agitated thin film drying;
c) drying at about 90-120° C. and
d) isolation of Form I of linezolid

The organic solvent used in steps (a) is selected from the group comprising of esters, alcohols, nitriles, ketones, ethers, amides, dialkylsulfoxide, chlorinated solvents or the mixtures thereof.

The inventors further discovered that the substantially solvent-free crystals of linezolid thus obtained in above mentioned process are remarkably stable as compared with the linezolid having residual solvent as per ICH guidelines. Substantially solvent-free crystals of linezolid, of the instant invention, are stable up to 12 months during stability study under different conditions of relative humidity (RH) and temperature (Table-1).

In the preferred embodiment ethyl acetate is used as crystallizing solvent for linezolid. It is understood that the ethyl acetate content of the substantially solvent-free crystals according to the present invention is not higher than about 1200 ppm; preferably not higher than about 1000 ppm, and for still better results, not higher than about 800 ppm.

TABLE-1
Determination of linezolid Form-II content in linezolid Form-I during stability study

| Name of the | Linezolid Form-I | | | | | | |
|---|---|---|---|---|---|---|---|
| product: | | | 1^{st} | 3^{rd} | 6^{th} | 9^{th} | 12^{th} |
| Condition | Tests | Initial | month | month | month | month | month |
| Temp-25 ± 2° C. | Polymorph | *ND | ND | ND | ND | ND | ND |
| RH-60 ± 5% | Status, | | | | | | |
| | XRD | | | | | | |
| Temp-40 ± 2° C. | | ND | ND | ND | ND | ND | ND |
| RH-75 ± 5% | | | | | | | |
| Temp-2-8° C. | | ND | ND | ND | ND | ND | ND |

*ND—Not Detected

The present invention also encompasses the process for the preparation of more than 99.9% pure linezolid free from bislinezolid the process comprising removal of N,N-bis[[(5S)-3-[3-fluoro-4-(4-morpholinyl)phenyl]-2-oxo-5-oxazolidinyl]methyl]amine (amino dimer impurity) compound of formula III from the reaction mixture of (S)-[[N-3-(3-Fluoro-4-morpholinylphenyl)-2-oxo-5-oxazolidinyl]methyl] amine p-TSA salt in hydrochloric acid at pH to 4.5-4.7 by washing the solution with ester solvent.

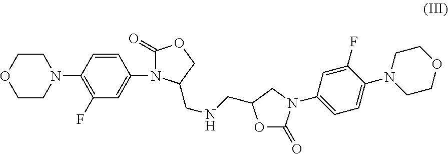 [SMILES: O=C1OC(CNCC2COC(=O)N2c2ccc(N3CCOCC3)c(F)c2)CN1c1ccc(N2CCOCC2)c(F)c1]

The present invention also encompasses the process for the preparation of more than 99.9% pure linezolid free from bislinezolid the process comprising removal of N,N-bis[[(5S)-3-[3-fluoro-4-(4-morpholinyl)phenyl]-2-oxo-5-oxazolidinyl]methyl]amine (amino dimer impurity) compound of formula III from the reaction mixture of (S)-[[N-3-(3-Fluoro-4-morpholinylphenyl)-2-oxo-5-oxazolidinyl]methyl] amine p-TSA salt in hydrochloric acid at pH to 4.5-4.7 by washing the solution with ethyl acetate.

The present invention also encompasses the process for the preparation of more than 99.9% pure linezolid free from N-(2-{[3-fluoro-4-(morpholine-4-yl) phenyl]amino}-1-hydroxyethyl)acetamide compound of formula IV from the reaction mixture of (S)-[[N-3-(3-Fluoro-4-morpholinylphenyl)-2-oxo-5-oxazolidinyl]methyl] amine p-TSA salt, acetic anhydride and water at pH to 7-7.5 by extraction of the reaction mixture using chlorinated solvent.

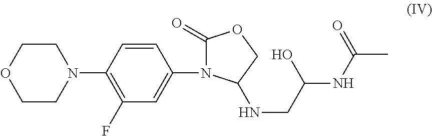 [SMILES: CC(=O)NC(O)CNC1COC(=O)N1c1ccc(N2CCOCC2)c(F)c1]

The present invention also encompasses the process for the preparation of more than 99.9% pure linezolid free from N-(2-{[3-fluoro-4-(morpholine-4-yl) phenyl]amino}-1-hydroxyethyl)acetamide compound of formula IV from the reaction mixture of (S)-[[N-3-(3-Fluoro-4-morpholinylphenyl)-2-oxo-5-oxazolidinyl]methyl] amine p-TSA salt, acetic anhydride and water at pH to 7-7.5 by extraction of the reaction mixture using dichloro methane solvent.

The substantially solvent-free crystals of linezolid obtained above can be processed into the desired dosage forms by the routine pharmaceutical procedures and be put to use as medicines, for example, antimicrobial agent. For substantially solvent-free crystals of linezolid, the procedures described in the Reference Examples, for instance, can be employed.

In another aspect there is provided a pharmaceutical composition that includes a therapeutically effective amount of linezolid or salts thereof according to the process of the present invention and one or more pharmaceutically acceptable carriers, excipients or diluents.

In yet another aspect there is provided a use of a pharmaceutical composition that includes a therapeutically effective amount of linezolid or salts thereof according to the process of the present invention and one or more pharmaceutically acceptable carriers, excipients or diluents to treat conditions in a subject, in need thereof such as antibacterial agent.

In another aspect there is provided a pharmaceutical composition that includes a therapeutically effective amount of enantiomeric pure linezolid Form-I according to the process of the present invention and one or more pharmaceutically acceptable carriers, excipients or diluents.

In yet another aspect there is provided a use of a pharmaceutical composition that includes a therapeutically effective amount of enantiomeric pure linezolid Form-I according to the process of the present invention and one or more pharmaceutically acceptable carriers, excipients or diluents to treat conditions in a subject, in need thereof such as antibacterial agent.

The present invention is further illustrated by the following examples, which are provided merely to be exemplary of the invention and do not limit the scope of the invention. Certain modifications and equivalents will be apparent to those skilled in the art and are intended to be included within the scope of the present invention.

### BRIEF DESCRIPTION OF THE FIGURES

FIG. 1 shows a representative X-ray diffraction pattern of enantiomeric pure linezolid Form-I

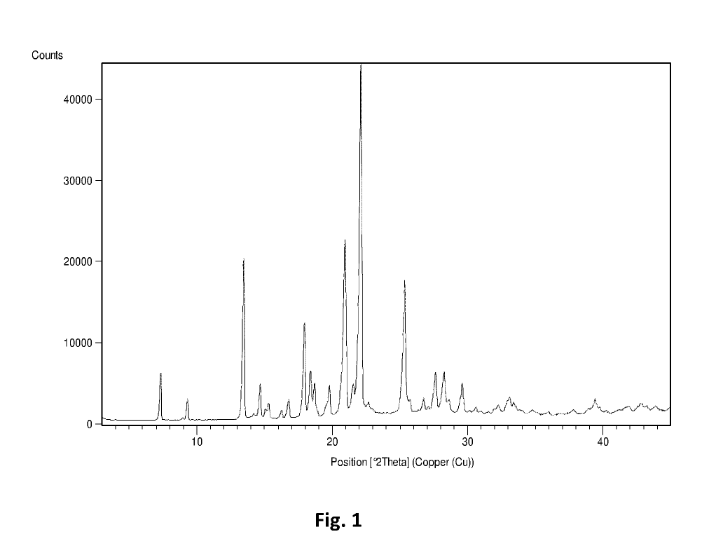

### EXAMPLES

### Example-1

### Preparation of (R)—[N-3-(3-fluoro-4-morpholinyl phenyl)-2-oxo-5-oxazolidinyl]methanol

To a stirred solution of benzyl (3-fluoro-4-morpholinyl)carbamate (100 g, 0.303 moles) in THF (800 mL) at −78° C. was added n-butyl lithium solution (1.6 M in hexanes, 208 mL, 0.337 moles) in 30 min followed by stirring for 2 hr. The solution of R-glycidyl butyrate (53 g, 0.368 moles) in THF (100 mL) was then added in 30 min and the mixture was stirred at −78° C. for 2 hr. The reaction mass was then stirred at room temperature for 12 hr, followed by quenched with ammonium chloride solution (90 g, 0.84 moles in 300 mL demineralised water) followed by addition of demineralised water (50 mL). The reaction mixture was stirred at room temperature for 30 min. The aqueous and organic layers were separated. The aqueous layer was extracted with ethyl acetate (2×250 mL). The combined ethyl acetate layer was recovered under vacuum at 50-55° C. and the main organic layer was charged to the residue and recovered under vacuum at 50-55° C. The obtained residue was stirred in ethyl acetate (700 mL) at 50° C., cooled to 40° C. The residue was filtered through hyflo and washed with ethyl acetate (200 mL). The combined ethyl acetate layer was cooled to 30° C. n-Hexane (300 mL) was added to the ethyl acetate solution at 25-30° C. The resulting mixture was stirred for 12 hr and then filtered.

Enantiomeric Purity: S-isomer 0.58%.

### Example-2

### Preparation of (R)—[N-3-(3-fluoro-4-morpholinyl phenyl)-2-oxo-5-oxazolidinyl]methanol

To a stirred solution of benzyl (3-fluoro-4-morpholinyl)carbamate (100 g, 0.303 moles) in THF (800 mL) at −78° C. was added n-butyl lithium solution (1.6 M in hexanes, 208.5 mL, 0.337 moles) in 30 min followed by stirring for 2 hr. The solution of R-glycidyl butyrate (53.0 g, 0.368 moles) in THF (100 mL) was added in 30 min and continued stirring at −78° C. for next 2 hr. The reaction mass was then stirred at room temperature for 12 hr. The solution of ammonium chloride (90.0 g, 0.84 moles in 300 mL demineralised water) was added followed by addition of demineralized water (50 mL). The reaction mixture was stirred at room temperature 30 min. The aqueous and organic layers were separated. The aqueous layer was extracted with ethyl acetate (2×250 mL). The combined ethyl acetate layer was recovered under vacuum at 50-55° C. and the main organic layer was charged to the residue and recovered under vacuum at 50-55° C. The obtained residue was stirred in ethyl acetate (700 mL) at 50° C., cooled to 40° C. The residue was filtered through hyflo and washed with ethyl acetate (200 mL). The moisture content of the combined ethyl acetate layer was adjusted to 1.03% by means of adding demineralised water (6 mL) and then cooled to 30° C. n-Hexane (600 mL) was added to the above ethyl acetate solution at 25-30° C. and stirred for 2 hr and a small sample was filtered and analyzed.

Enantiomeric Purity: S-isomer 0.50%.

### Example-3

### Preparation of (R)—[N-3-(3-fluoro-4-morpholinyl phenyl)-2-oxo-5-oxazolidinyl]methanol

To a suspension of benzyl (3-fluoro-4-morpholinyl)carbamate (50 .g, 0.152 moles) in THF (400 mL) at −78° C. was added n-butyl lithium solution (1.6 M in hexanes, 104 mL, 0.167 moles) in 30 min followed by stirring for 2 hr. The solution of R-glycidyl butyrate (26.2 g, 0.182 moles) in THF (50 mL) was then added in 30 min and continued stirring at −78° C. for 2 hr. The reaction mixture was stirred at room temperature for 12 hr and quenched by ammonium chloride solution (45.0 g, 0.84 moles in 150 mL demineralised water) followed by addition of demineralised water (25 mL). The reaction mixture was stirred for 30 min. The both aqueous and organic layers were separated. The aqueous layer was extracted with ethyl acetate (2×125 mL). The combined ethyl acetate layer was recovered under vacuum at 50-55° C. and then main organic layer was charged to the residue and recovered under vacuum at 50-55° C. The obtained residue was stirred in ethyl acetate (350 mL) at 50° C., cooled to 40° C. and filtered through hyflo and washed with ethyl acetate (100 mL). The moisture content of the combined ethyl acetate layer was adjusted to 0.28% by means of adding demineralised water (1.5 mL) and cooled to 30° C. n-Hexane (300 mL) was added to the ethyl acetate solution at 25-30° C. and stirred for 12 hr, filtered the solid and dried at 50-55° C. for 18 hr. The mother liquor was concentrated to dryness under vacuum at 50° C. and crystallized from a mixture of ethyl acetate (150 mL) and n-hexane (150 mL) to get the 2^{nd} crop of (R)—[N-3-(3-fluoro-4-morpholinyl phenyl)-2-oxo-5-oxazolidinyl]methanol, which matches with the 1^{st} crop in all respect to provide 28.7 g material in a combined.

Enantiomeric Purity: S-isomer 0.02%.

Percentage Yield: 64%

### Example-4

### Preparation of (R)—[N-3-(3-fluoro-4-morpholinyl phenyl)-2-oxo-5-oxazolidinyl]methanol

To a stirred solution of benzyl (3-fluoro-4-morpholinyl)carbamate (50 g, 0.152 moles) in THF (400 mL) at −78° C. was added n-butyl lithium solution (1.6 M in hexanes, 104 mL, 0.167 moles) in 30 min followed by stirring for 2 hr. The solution of R-glycidyl butyrate (26.2 g, 0.182 moles) in THF (50 mL) was added in 30 min and continued stirring at −78° C. for 2 hr. The reaction mass was then stirred at room temperature for 12 hr and quenched with ammonium solution (45.0 g, 0.84 moles in 150 mL demineralised water) followed by addition of demineralised water (25 mL). The reaction mixture was stirred for 30 min. The both aqueous and organic layers were separated. The aqueous layer was extracted with ethyl acetate (2×125 mL). The combined ethyl acetate layer was recovered under vacuum at 50-55° C. and the main organic layer was charged to the residue and recovered under vacuum at 50-55° C. The obtained residue was stirred in ethyl acetate (350 mL) at 50° C., cooled to 40° C. and filtered through hyflo and washed with ethyl acetate (100 mL). The moisture content in the combined ethyl acetate layer was adjusted to 0.28% by adding demineralised water (1.5 mL) and then cooled to 30° C. Cyclohexane (225 mL) was charged to the above ethyl acetate solution at 25-30° C. and stirred for 5 hr, filtered the solid and dried at 50-55° C. for 18 hr. The mother liquor was concentrated to dryness under vacuum at 50° C. and crystallized from a mixture of ethyl acetate (150 mL) and cyclohexane (150 mL) to get the 2^{nd} crop of (R)—[N-3-(3-fluoro-4-morpholinylphenyl)-2-oxo-5-oxazolidinyl]methanol which matches with the 1^{st} crop in all respect to provide 34 g material with a combined 76% yield.

Enantiomeric Purity: S-isomer 0.05%.

Percentage Yield: 76%

### Example-5

### Preparation of (R)—[N-3-(3-fluoro-4-morpholinyl phenyl)-2-oxo-5-oxazolidinyl]methanol

To a stirred solution of benzyl (3-fluoro-4-morpholinyl)carbamate (100 g, 0.303 moles) in THF (800 mL) at −78° C. was added n-butyl lithium solution (1.6 M in hexanes, 208.5 mL, 0.337 moles) in 30 min followed by stirring for 2 hr. The solution of R-glycidyl butyrate (53 g, 0.368 moles) in THF (100 mL) was then added in 30 min and continued stirring at −78° C. for next 2 hr. The reaction mass was stirred at room temperature for 12 hr and quenched by ammonium chloride solution (90 g, 0.84 moles in 300 mL demineralised water) followed by addition of demineralised water (50 mL). The reaction mixture was stirred at room temperature for 30 min. The both aqueous and organic layers were separated. The aqueous layer was extracted with ethyl acetate (2×250 mL). The combined ethyl acetate layer was recovered under vacuum at 50-55° C. and the main organic layer was charged to the residue and recovered under vacuum at 50-55° C. The obtained residue was dissolved in ethyl acetate (700 mL) at 50° C., cooled to 40° C. and filtered through hyflo and washed with ethyl acetate (200 mL). The moisture content of the combined ethyl acetate layer was adjusted to 0.42% by adding demineralised water (1.0 mL) and then cooled to 30° C. Cyclohexane (600 mL) was added to the above ethyl acetate solution at 2530° C. and stirred for 12 hr, filtered the solid and dried at 50-55° C. for 18 hr. The mother liquor was concentrated under vacuum at 50° C. and crystallized from a mixture of ethyl acetate (300 mL) and cyclohexane (300 mL) to get the 2^{nd} crop of (R)—[N-3-(3-fluoro-4-morpholinylphenyl)-2oxo-5-oxazolidinyl]methanol which matches with the 1st crop in all respect and to provide 65 g combined material.

Enantiomeric Purity: S-isomer 0.05%.

Percentage Yield: 73%

### Example-6

### Preparation of (R)—[N-3-(3-fluoro-4-morpholinyl phenyl)-2-oxo-5-oxazolidinyl]methanol

To a stirred solution of benzyl (3-fluoro-4-morpholinyl)carbamate (20 g, 0.0606 moles) in THF (160 mL) at −78° C. was added n-butyl lithium solution (1.6 M in hexanes, 41.7 mL, 0.0674 moles) in 30 min followed by stirring for 2 hr. The solution of R-glycidyl butyrate (having 2.3% S-isomer) (10.6 g, 0.0736 moles) in THF (20 mL) was then added in 30 min and continued stirring at −78° C. for next 2 hr. The reaction mass was stirred at room temperature for 12 hr and quenched by ammonium chloride solution (18 g, 0.168 moles in 60 mL demineralised water) followed by addition of demineralised water (10 mL). The reaction mixture was stirred at room temperature for 30 min. The both aqueous and organic layers were separated. The aqueous layer was extracted with ethyl acetate (2×50 mL). The combined ethyl acetate layer was recovered under vacuum at 50-55° C. and the main organic layer was charged to the residue and recovered under vacuum at 50-55° C. The obtained residue was dissolved in ethyl acetate (140 mL) at 50° C., cooled to 40° C. and filtered through hyflo and washed with ethyl acetate (40 mL). The moisture content of the combined ethyl acetate layer was adjusted to 0.37% by adding demineralised water (0.75 mL) and then cooled to 30° C. Cyclohexane (120 mL) was added to the above ethyl acetate solution at 25-30° C. and stirred for 12 hr, filtered the solid and dried at 50-55° C. for 16 hr. The mother liquor was concentrated under vacuum at 50° C. and crystallized from a mixture of ethyl acetate (60 mL) and cyclohexane (60 mL) to get the 2^{nd} crop of (R)—[N-3-(3-fluoro-4-morpholinylphenyl)-2-oxo-5-oxazolidinyl]methanol which matches with the 1^{st} crop in all respect and to provide 12.8 g combined material.

Enantiomeric Purity: S-isomer 0.05%.

HPLC Purity: 99.69%

Percentage Yield: 73%

### Example-7

### Preparation of (R)—[N-3-(3-Fluoro-4-morpholinylphenyl)-2-oxo-5-oxazolidinyl] methanol

To a solution of N-(3-Fluoro-4-morpholin-4-ylphenyl)carbamic acid benzyl ester (200 g, 0.606 mol) in THF (1600 mL) under nitrogen at −78° C. was added n-butyllithium (416.7 mL, 1.6 M in hexane, 0.666 mol, 1.1 mol eq) over 1.5 h. The reaction mixture was stirred at −78° C. for 2 h, then a solution of R-(−)-glycidyl butyrate (104.7 g, 0.727 mol, 1.2 mol eq) in THF (200 mL) was added at −78° C. over 1 h. After stirring at −78° C. for 2 h, the reaction mass was warmed to room temperature and stirred for overnight. To the resulting thick slurry is then added saturated ammonium chloride (690 mL) followed by water (100 mL). After stirring at room temperature for 10 min THF layer was separated, aqueous layer was extracted with ethyl acetate (2×500 mL). The combined ethyl acetate layer was concentrated under vacuum at 50-55° C. to get a residue in which THF layer was added and concentrated completely under vacuum at 50-55° C. Thus obtained solid mass was cooled to room temperature and ethyl acetate (1600 mL) was added. The mixture was heated to 55-60° C. and kept stirring for 30 min. The mixture was cooled to 40-42° C., filtered over hyflo and then washed the bed with ethyl acetate (200 mL). The moisture content in the combined ethyl acetate layer was adjusted to 0.29% by adding demineralised water and then cooled to 30° C. n-Hexane (1200 mL) was charged to the above ethyl acetate solution at 25-30° C. and stirred for 12 hr, filtered the solid, washed with a mixture (1:1) of ethyl acetate and n-hexane (2×200 mL) and dried under vacuum at 50-55° C. for 16 hr. The mother liquor was concentrated to dryness under vacuum at 50° C. and crystallized from a mixture of ethyl acetate (600 mL) and cyclohexane (600 mL) to get the 2^{nd} crop of (R)—[N-3-(3-fluoro-4-morpholinylphenyl)-2-oxo-5-oxazolidinyl]methanol which matches with the 1^{st} crop in all respect to provide 142 g material with a combined 80% yield.

Percentage Yield: 80%

### Example-8

### Preparation of (R)—[N-3-(3-Fluoro-4-morpholinylphenyl)-2-oxo-5-oxazolidinyl]methyl 4-methylbenzenesulfonate

To a cold (0-5° C.) solution of (R)—[N-3-(3-Fluoro-4-morpholinylphenyl)-2-oxo-5-oxazolidinyl] methanol (120 g, 0.405 mol) and p-toluenesulfonyl chloride (115.9 g, 0.608 mol) in DCM (720 mL) was added triethylamine (69.6 g, 0.689 mol) over 50 min at 5-10° C. The solution was warmed to room temperature and stirred for 18 h. After the completion of reaction, water (600 mL) was added and stirred for 10 min. DCM layer was collected and concentrated under atmospheric pressure at 35-40° C. till ˜240 mL, (2 volume) left in the flask. Methanol (600 mL) was added and stirred for a while and ˜120 mL (1 volume) solvent was recovered at atmospheric pressure at 60-65° C. Methanol (1200 mL) was added in the reaction mass and allowed to cool to room temperature with stirring. Then it was stirred for 45 min. The solid was filtered, washed with methanol (2×300 mL) and then dried under vacuum at 50-55° C. for overnight to obtain the title compound (166 g) with 91% yield.

Percentage Yield: 91%

### Example-9

### Preparation of (S)—N-[[3-(3-Fluoro-4-morpholinylphenyl)-2-oxo-5-oxazolidinyl]methyl]amine p-TSA salt

A suspended solution of (R)-[[N-3-(3-Fluoro-4-morpholinylphenyl)-2-oxo-5-oxazolidinyl] methyl] p-toluenesulfonate (100 g, 0.222 mol) in a mixture (1:1:1) of THF/IPA/aqueous ammonium hydroxide (1500 mL) was heated in a autoclave to 80-85° C. and kept stirring 80-85° C. for 24 h. After completion of reaction it was cooled to room temperature. The reaction mass was concentrated to dryness under vacuum at 50-55° C. Residual moisture was removed by using IPA (200 mL) followed by recovery under vacuum at 50-55° C. twice till to get moisture content <1%. To the resulting residue IPA (300 mL) was added and stirred at 60-65° C. for 2 h. After cooling to room temperature the solid was filtered, washed with IPA (2×100 mL) and dried under vacuum at 50-55° C. for overnight. To the above obtained solid DCM (500 mL) was added and heated to reflux for 1 h. After cooling to room temperature the suspension was stirred for 1 h. The solid mass was filtered, washed with DCM (2×100 mL) and then dried under vacuum at 50-55° C. for overnight to obtain the title compound (88 g) with 84% yield.

Percentage Yield: 84%

### Example-10

### Preparation of (S)—N-[[3-(3-Fluoro-4-morpholinylphenyl)-2-oxo-5-oxazolidinyl] methyl acetamide, linezolid (Form-I)

To a suspension of (S)-[[N-3-(3-Fluoro-4-morpholinylphenyl)-2-oxo-5-oxazolidinyl]methyl] amine p-TSA salt (60 g, 0.128 mol) in water (600 mL) was added 6N hydrochloric acid (60 mL) at room temperature to adjust its pH to ˜0.5. Ethyl acetate (600 mL) was added in the above solution and then readjusted its pH to 4.5-4.7 using 10% sodium hydroxide solution (130 mL). After separating ethyl acetate layer aqueous layer was washed with ethyl acetate (2×300 mL). To the acidic aqueous layer were added DCM (600 mL) and acetic anhydride (26.11 g, 0.256 mol). The biphasic reaction mixture was adjusted its pH to 4.5-4.7 using 10% sodium hydroxide solution (130 mL) and stirred at room temperature for 3 h at this pH. After completion of reaction pH of the reaction mixture was raised to ˜7-7.5 using 10% sodium hydroxide solution (100 mL). Separating DCM layer, aqueous layer was extracted with DCM (600 mL). The combined DCM layer was washed with water (2×300 mL). DCM layer was concentrated under vacuum at 35-40° C. completely and kept under vacuum at 35-40° C. for 1 h. The obtained solid mass was dissolved in ethyl acetate (1020 mL) at 70-75° C. and cooled to 60° C. The resulting solution was filtered through hyflo bed and washed with ethyl acetate (180 mL). To a cold (−15° C.) ethyl acetate (175 mL) was added the above combined ethyl acetate solution of crude linezolid at −15 to 10° C. in 5-10 min and stirred for 10 min without further cooling. The suspended solution was then cooled down to −15° C. and stirred at −15 to −10° C. for 2 h. The solid mass was filtered, kept under suction for 1 h and then dried under vacuum at 50-55° C. for overnight to obtain the title compound (27 g) with 63% yield.

### Yield: 63%

### Polymorph: Form-I

Polymorphic impurity: Below detection limit (slow scan count 495)

### Enantiomer Purity: R-isomer 0.03%

### Example-11

### Preparation of (R)—[N-3-(3-Fluoro-4-morpholinylphenyl)-2-oxo-5-oxazolidinyl]methyl 4-methylbenzenesulfonate

To a cold (0-5° C.) solution of (R)—[N-3-(3-Fluoro-4-morpholinylphenyl)-2-oxo-5-oxazolidinyl]methanol (120 g, 0.405 mol) and p-toluenesulfonyl chloride (115.9 g, 0.608 mol) in DCM (720 mL) was added triethylamine (69.6 g, 0.689 mol) over 50 min at 5-10° C. The solution was warmed to room temperature and stirred for 18 h. After the completion of reaction, water (600 mL) was added and stirred for 10 min. DCM layer was collected and concentrated under atmospheric pressure at 35-40° C. till ˜240 mL, (2 volume) left in the flask. Methanol (600 mL) was added and stirred for a while and ˜120 mL (1 volume) solvent was recovered at atmospheric pressure at 60-65° C. Methanol (1200 mL) was added in the reaction mass and allowed to cool to room temperature with stirring. Then it was stirred for 45 min. The solid was filtered, washed with methanol (2×300 mL) and then dried under vacuum at 50-55° C. for overnight to obtain the title compound (165 g) with 90% yield.

### Example-12

### Preparation of (S)—N-[[3-(3-Fluoro-4-morpholinylphenyl)-2-oxo-5-oxazolidinyl]methyl]amine p-TSA salt

A suspended solution of (R)-[[N-3-(3-Fluoro-4-morpholinylphenyl)-2-oxo-5-oxazolidinyl] methyl] p-toluenesulfonate (100 g, 0.222 mol) in a mixture (1:1:1) of THF/IPA/aqueous ammonium hydroxide (1500 mL) was heated in a autoclave to 80-85° C. and kept stirring 80-85° C. for 24 h. After completion of reaction it was cooled to room temperature. The reaction mass was concentrated to dryness under vacuum at 50-55° C. Residual moisture was removed by using IPA (200 mL) followed by recovery under vacuum at 50-55° C. twice till to get moisture content <1%. To the resulting residue IPA (300 mL) was added and stirred at 60-65° C. for 2 h. After cooling to room temperature the solid was filtered, washed with IPA (2×100 mL) and dried under vacuum at 50-55° C. for overnight. To the above obtained solid DCM (500 mL) was added and heated to reflux for 1 h. After cooling to room temperature the suspension was stirred for 1 h. The solid mass was filtered, washed with DCM (2×100 mL) and then dried under vacuum at 50-55° C. for overnight to obtain the title compound (87.7 g) with a 84% yield.

### Example-13

### Preparation of (S)—N-[[3-(3-Fluoro-4-morpholinylphenyl)-2-oxo-5-oxazolidinyl]methyl acetamide, linezolid (Form-I)

To a suspension of (S)-[[N-3-(3-Fluoro-4-morpholinylphenyl)-2-oxo-5-oxazolidinyl]methyl] amine p-TSA salt (60 g, 0.128 mol) obtained from example 3 in water (600 mL) was added 6N hydrochloric acid (60 mL) at room temperature to adjust its pH to ˜0.5. Ethyl acetate (600 mL) was added in the above solution and then readjusted its pH to 4.5-4.7 using 10% sodium hydroxide solution (130 mL). After separating ethyl acetate layer aqueous layer was washed with ethyl acetate (2×300 mL). To the acidic aqueous layer were added DCM (600 mL) and acetic anhydride (26.11 g, 0.256 mol). The biphasic reaction mixture was adjusted its pH to 4.5-4.7 using 10% sodium hydroxide solution (130 mL) and stirred at room temperature for 3 h at this pH. After completion of reaction pH of the reaction mixture was raised to ˜7-7.5 using 10% sodium hydroxide solution (100 mL). Separating DCM layer, aqueous layer was extracted with DCM (600 mL). The combined DCM layer was washed with water (2×300 mL). DCM layer was concentrated under vacuum at 35-40° C. completely and kept under vacuum at 35-40° C. for 1 h. The obtained solid mass was dissolved in ethyl acetate (1020 mL) at 70-75° C. and cooled to 60° C. The resulting solution was filtered through hyflo bed and washed with ethyl acetate (180 mL). To a cold (−15° C.) ethyl acetate (175 mL) was added the above combined ethyl acetate solution of crude linezolid at −15 to 10° C. in 5-10 min and stirred for 10 min without further cooling. The suspended solution was then cooled down to −15° C. and stirred at −15 to −10° C. for 2 h. The solid mass was filtered, kept under suction for 1 h and then dried under vacuum at 50-55° C. for overnight to obtain the title compound (33 g) with 76% yield.

### Yield: 76%

### Polymorph: Form-I

Polymorphic impurity: Below detection limit (slow scan count 290)

### Enantiomer Purity: R-isomer 0.04%

### Example-13

### Preparation of (R)—[N-3-(3-Fluoro-4-morpholinylphenyl)-2-oxo-5-oxazolidinyl] methyl 4-methylbenzenesulfonate

To a cold (0-5° C.) solution of (R)—[N-3-(3-Fluoro-4-morpholinylphenyl)-2-oxo-5-oxazolidinyl]methanol (120 g, 0.405 mol) and p-toluenesulfonyl chloride (115.9 g, 0.608 mol) in DCM (720 mL) was added triethylamine (69.6 g, 0.689 mol) over 50 min at 5-10° C. The solution was warmed to room temperature and stirred for 18 h. After the completion of reaction, water (600 mL) was added and stirred for 10 min. DCM layer was collected and concentrated under atmospheric pressure at 35-40° C. till ˜240 mL, (2 volume) left in the flask. Methanol (600 mL) was added and stirred for a while and ˜120 mL (1 volume) solvent was recovered at atmospheric pressure at 60-65° C. Methanol (1200 mL) was added in the reaction mass and allowed to cool to room temperature with stirring. Then it was stirred for 45 min. The solid was filtered, washed with methanol (2×300 mL) and then dried under vacuum at 50-55° C. for overnight to obtain the title compound (167 g) with 92% yield.

### Example-14

### Preparation of (S)—N-[[3-(3-Fluoro-4-morpholinylphenyl)-2-oxo-5-oxazolidinyl] methyl] amine p-TSA salt

A suspended solution of (R)-[[N-3-(3-Fluoro-4-morpholinylphenyl)-2-oxo-5-oxazolidinyl] methyl] p-toluenesulfonate (100 g, 0.222 mol) in a mixture (1:1:1) of THF/IPA/aqueous ammonium hydroxide (1500 mL) was heated in a autoclave to 80-85° C. and kept stirring 80-85° C. for 24 h. After completion of reaction it was cooled to room temperature. The reaction mass was concentrated to dryness under vacuum at 50-55° C. Residual moisture was removed by using IPA (200 mL) followed by recovery under vacuum at 50-55° C. twice till to get moisture content <1%. To the resulting residue IPA (300 mL) was added and stirred at 60-65° C. for 2 h. After cooling to room temperature the solid was filtered, washed with IPA (2×100 mL) and dried under vacuum at 50-55° C. for overnight. To the above obtained solid DCM (500 mL) was added and heated to reflux for 1 h. After cooling to room temperature the suspension was stirred for 1 h. The solid mass was filtered, washed with DCM (2×100 mL) and then dried under vacuum at 50-55° C. for overnight to obtain the title compound (87 g) with a 84% yield.

### Example-15

### Preparation of (S)—N-[[3-(3-Fluoro-4-morpholinylphenyl)-2-oxo-5-oxazolidinyl]methyl acetamide, linezolid (Form-I)

To a suspension of (S)-[[N-3-(3-Fluoro-4-morpholinylphenyl)-2-oxo-5-oxazolidinyl]methyl] amine p-TSA salt (60 g, 0.128 mol) in water (600 mL) was added 6N hydrochloric acid (60 mL) at room temperature to adjust its pH to ˜0.5. Ethyl acetate (600 mL) was added in the above solution and then readjusted its pH to 4.5-4.7 using 10% sodium hydroxide solution (130 mL). After separating ethyl acetate layer aqueous layer was washed with ethyl acetate (2×300 mL). To the acidic aqueous layer were added DCM (600 mL) and acetic anhydride (26.11 g, 0.256 mol). The biphasic reaction mixture was adjusted its pH to 4.5-4.7 using 10% sodium hydroxide solution (130 mL) and stirred at room temperature for 3 h at this pH. After completion of reaction pH of the reaction mixture was raised to ˜7-7.5 using 10% sodium hydroxide solution (100 mL). Separating DCM layer, aqueous layer was extracted with DCM (600 mL). The combined DCM layer was washed with water (2×300 mL). DCM layer was concentrated under vacuum at 35-40° C. completely and kept under vacuum at 35-40° C. for 1 h. The obtained solid mass was dissolved in ethyl acetate (1020 mL) at 70-75° C. and cooled to 60° C. The resulting solution was filtered through hyflo bed and washed with ethyl acetate (180 mL). To a cold (−15° C.) ethyl acetate (175 mL) was added the above combined ethyl acetate solution of crude linezolid at −15 to 10° C. in 5-10 min and stirred for 10 min without further cooling. The suspended solution was then cooled down to −15° C. and stirred at −15 to −10° C. for 2 h. The solid mass was filtered, kept under suction for 1 h and then dried under vacuum at 50-55° C. for overnight to obtain the title compound (34.5 g) with 80% yield.

### Yield: 80%

### Polymorph: Form-I

Polymorphic impurity: Below detection limit (slow scan count 629)

### Enantiomer Purity: R-isomer 0.04%

### Example-16

### Preparation of (R)—[N-3-(3-Fluoro-4-morpholinylphenyl)-2-oxo-5-oxazolidinyl]methyl 4-methylbenzenesulfonate

To a cold (0-5° C.) solution of (R)—[N-3-(3-Fluoro-4-morpholinylphenyl)-2-oxo-5-oxazolidinyl]methanol (120 g, 0.405 mol) and p-toluenesulfonyl chloride (115.9 g, 0.608 mol) in DCM (720 mL) was added triethylamine (69.6 g, 0.689 mol) over 50 min at 5-10° C. The solution was warmed to room temperature and stirred for 18 h. After the completion of reaction, water (600 mL) was added and stirred for 10 min. DCM layer was collected and concentrated under atmospheric pressure at 35-40° C. till ˜240 mL, (2 volume) left in the flask. Methanol (600 mL) was added and stirred for a while and ˜120 mL (1 volume) solvent was recovered at atmospheric pressure at 60-65° C. Methanol (1200 mL) was added in the reaction mass and allowed to cool to room temperature with stirring. Then it was stirred for 45 min. The solid was filtered, washed with methanol (2×300 mL) and then dried under vacuum at 50-55° C. for overnight to obtain the title compound (165.5 g) with 91% yield.

Percentage Yield: 91%

### Example-17

### Preparation of (S)—N-[[3-(3-Fluoro-4-morpholinylphenyl)-2-oxo-5-oxazolidinyl]methyl]amine p-TSA salt

A suspended solution of (R)-[[N-3-(3-Fluoro-4-morpholinylphenyl)-2-oxo-5-oxazolidinyl] methyl] p-toluenesulfonate (100 g, 0.222 mol) in a mixture (1:1:1) of THF/IPA/aqueous ammonium hydroxide (1500 mL) was heated in a autoclave to 80-85° C. and kept stirring 80-85° C. for 24 h. After completion of reaction it was cooled to room temperature. The reaction mass was concentrated to dryness under vacuum at 50-55° C. Residual moisture was removed by using IPA (200 mL) followed by recovery under vacuum at 50-55° C. twice till to get moisture content <1%. To the resulting residue IPA (300 mL) was added and stirred at 60-65° C. for 2 h. After cooling to room temperature the solid was filtered, washed with IPA (2×100 mL) and dried under vacuum at 50-55° C. for overnight. To the above obtained solid DCM (500 mL) was added and heated to reflux for 1 h. After cooling to room temperature the suspension was stirred for 1 h. The solid mass was filtered, washed with DCM (2×100 mL) and then dried under vacuum at 50-55° C. for overnight to obtain the title compound (85 g) with 82% yield.

Percentage Yield: 82%

### Example-18

### Preparation of (S)—N-[[3-(3-Fluoro-4-morpholinylphenyl)-2-oxo-5-oxazoli dinyl] methyl acetamide, linezolid (Form-I)

To a suspension of (S)-[[N-3-(3-Fluoro-4-morpholinylphenyl)-2-oxo-5-oxazolidinyl]methyl] amine p-TSA salt (60 g, 0.128 mol) in water (600 mL) was added 6N hydrochloric acid (60 mL) at room temperature to adjust its pH to ˜0.5. Ethyl acetate (600 mL) was added in the above solution and then readjusted its pH to 4.5-4.7 using 10% sodium hydroxide solution (130 mL). After separating ethyl acetate layer aqueous layer was washed with ethyl acetate (2×300 mL). To the acidic aqueous layer were added DCM (600 mL) and acetic anhydride (26.11 g, 0.256 mol). The biphasic reaction mixture was adjusted its pH to 4.5-4.7 using 10% sodium hydroxide solution (130 mL) and stirred at room temperature for 3 h at this pH. After completion of reaction pH of the reaction mixture was raised to ˜7-7.5 using 10% sodium hydroxide solution (100 mL). Separating DCM layer, aqueous layer was extracted with DCM (600 mL). The combined DCM layer was washed with water (2×300 mL). DCM layer was concentrated under vacuum at 35-40° C. completely and kept under vacuum at 35-40° C. for 1 h. The obtained solid mass was dissolved in ethyl acetate (1020 mL) at 70-75° C. and cooled to 60° C. The resulting solution was filtered through hyflo bed and washed with ethyl acetate (180 mL). To a cold (−15° C.) ethyl acetate (175 mL) was added the above combined ethyl acetate solution of crude linezolid at −15 to 10° C. in 5-10 min and stirred for 10 min without further cooling. The suspended solution was then cooled down to −15° C. and stirred at −15 to −10° C. for 2 h. The solid mass was filtered, kept under suction for 1 h and then dried under vacuum at 50-55° C. for overnight to obtain the title compound (34.5 g) with 80% yield.

### Yield: 80%

### Polymorph: Form-I

Polymorphic impurity: Below detection limit (slow scan count 290)

### Enantiomer Purity: R-isomer 0.04%

### Example-19

### Preparation of (S)—N-[[3-(3-Fluoro-4-morpholinylphenyl)-2-oxo-5-oxazoli dinyl] methyl acetamide, linezolid (Form-I)

To a suspension of (S)-[[N-3-(3-Fluoro-4-morpholinylphenyl)-2-oxo-5-oxazolidinyl]methyl] amine p-TSA salt (50 g, 0.107 mol) in water (500 mL) was added 6N hydrochloric acid (50 mL) at room temperature to adjust its pH to ˜0.5. Ethyl acetate (500 mL) was added in the above solution and then readjusted its pH to 4.5-4.7 using 10% sodium hydroxide solution (125 mL). After separating ethyl acetate layer aqueous layer was washed with ethyl acetate (2×250 mL). To the acidic aqueous layer were added DCM (500 mL) and acetic anhydride (21.84 g, 0.214 mol). The biphasic reaction mixture was adjusted its pH to 4.5-4.7 using 10% sodium hydroxide solution (100 mL) and stirred at room temperature for 3 h at this pH. After completion of reaction pH of the reaction mixture was raised to ˜7-7.5 using 10% sodium hydroxide solution (75 mL). Separating DCM layer, aqueous layer was extracted with DCM (500 mL). The combined DCM layer was washed with water (2×250 mL). DCM layer was concentrated under vacuum at 35-40° C. completely and kept under vacuum at 35-40° C. for 1 h. The obtained solid mass was dissolved in ethyl acetate (1250 mL) at 60-65° C. and cooled to 50° C. The resulting solution was filtered through 0.45 micron filter paper and washed with ethyl acetate (150 mL). The combined filtrate was cooled to −15° C. with occasional stirring, turbidity was appeared. N-Hexane (1050 mL) was added at −15 to 0° C. in 5-10 min. The suspended solution was then cooled down to −15° C. and stirred at −15 to −10° C. for 2 h. The solid mass was filtered, kept under suction for 1 h and then dried under vacuum at 50-55° C. for overnight to obtain the title compound (24.6 g) with 68% yield.

### Yield: 68%

### Polymorph: Form-I

Polymorphic impurity: Below detection limit (slow scan count 247)

### Enantiomer Purity: R-isomer 0.02%

### Example-20

### Preparation of (S)—N-[[3-(3-Fluoro-4-morpholinylphenyl)-2-oxo-5-oxazolidinyl]methyl acetamide, linezolid (Form-I)

To a suspension of (S)-[[N-3-(3-Fluoro-4-morpholinylphenyl)-2-oxo-5-oxazolidinyl]methyl] amine p-TSA salt (60 g, 0.128 mol) obtained in water (600 mL) was added 6N hydrochloric acid (60 mL) at room temperature to adjust its pH to ˜0.5. Ethyl acetate (600 mL) was added in the above solution and then readjusted its pH to 4.5-4.7 using 10% sodium hydroxide solution (130 mL). After separating ethyl acetate layer, aqueous layer was washed with ethyl acetate (2×300 mL). To the acidic aqueous layer were added DCM (600 mL) and acetic anhydride (26.11 g, 0.256 mol). The biphasic reaction mixture was adjusted its pH to 4.5-4.7 using 10% sodium hydroxide solution (130 mL) and stirred at room temperature for 3 h at this pH. After completion of reaction pH of the reaction mixture was raised to ˜7-7.5 using 10% sodium hydroxide solution (100 mL). Separating DCM layer, aqueous layer was extracted with DCM (600 mL). The combined DCM layer was washed with water (2×300 mL). DCM layer was concentrated under vacuum at 35-40° C. completely and kept under vacuum at 35-40° C. for 1 h. The obtained solid mass was dissolved in ethyl acetate (1800 mL) at 60-65° C. and cooled to 45° C. The resulting solution was filtered through 0.45 micron filter paper. The combined filtrate was cooled to −15° C. with occasional stirring, a turbidity was appeared in 10 min. Cyclohexane (1350 mL) was added at −15 to −10° C. in 20-30 min. The suspended solution was then cooled down to −15° C. and stirred at −15 to −10° C. for 2 h. The solid mass was filtered, kept under suction for 1 h and then dried under vacuum at 50-55° C. for overnight to obtain the title compound (31.2 g) with 72% yield.

### Yield: 72%

### Polymorph: Form-I

Polymorphic impurity: Below detection limit (slow scan count 219)

### Enantiomer Purity: R-isomer 0.04%

### Example-21

### Preparation of (S)—N-[[3-(3-Fluoro-4-morpholinylphenyl)-2-oxo-5-oxazoli dinyl]methyl acetamide, linezolid (Form-I)

To a suspension of (S)-[[N-3-(3-Fluoro-4-morpholinylphenyl)-2-oxo-5-oxazolidinyl]methyl] amine p-TSA salt (25 g, 0.0535 mol) in water (250 mL) was added 6N hydrochloric acid (25 mL) at room temperature to adjust its pH to ˜0.5. Ethyl acetate (250 mL) was added in the above solution and then readjusted its pH to 4.5-4.7 using 10% sodium hydroxide solution (63 mL). After separating ethyl acetate layer aqueous layer was washed with ethyl acetate (2×125 mL). To the acidic aqueous layer were added DCM (250 mL) and acetic anhydride (10.92 g, 0.107 mol). The biphasic reaction mixture was adjusted its pH to 4.5-4.7 using 10% sodium hydroxide solution (60 mL) and stirred at room temperature for 3 h at this pH. After completion of reaction pH of the reaction mixture was raised to ˜7-7.5 using 10% sodium hydroxide solution (40 mL). Separating DCM layer, aqueous layer was extracted with DCM (250 mL). The combined DCM layer was washed with water (2×125 mL). DCM layer was concentrated under vacuum at 35-40° C. completely and kept under vacuum at 40-45° C. for 1 h. The obtained solid mass was dissolved in ethyl acetate (750 mL) at 60-65° C. and cooled to 50° C. The resulting solution was filtered through 0.45 micron filter paper and washed with ethyl acetate (125 mL). The combined filtrate was cooled to −15° C. with occasional stirring, a turbidity was appeared. N-Heptane (875 mL) was added at −15 to 0° C. in 15-20 min. The suspended solution was then cooled down to −15° C. and stirred at −15 to −10° C. for 1 h. The solid mass was filtered, kept under suction for 1 h and then dried under vacuum at 50-55° C. for overnight to obtain the title compound (11 g) with 61% yield.

### Yield: 61%

### Polymorph: Form-I

Polymorphic impurity: Below detection limit (slow scan count not detected)

### Enantiomer Purity: R-isomer 0.01%

### Example-22

### Preparation of (S)—N-[[3-(3-Fluoro-4-morpholinylphenyl)-2-oxo-5-oxazolidinyl]methyl acetamide, linezolid (Form-I)

To a suspension of (S)-[[N-3-(3-Fluoro-4-morpholinylphenyl)-2-oxo-5-oxazolidinyl]methyl] amine p-TSA salt (10 g, 0.0214 mol) in water (100 mL) was added 6N hydrochloric acid (60 mL) at room temperature to adjust its pH to ˜0.5. Ethyl acetate (100 mL) was added in the above solution and then readjusted its pH to 4.5-4.7 using 10% sodium hydroxide solution (25 mL). After separating ethyl acetate layer, aqueous layer was washed with ethyl acetate (2×50 mL). To the acidic aqueous layer were added DCM (100 mL) and acetic anhydride (4.4 g, 0.0428 mol). The biphasic reaction mixture was adjusted its pH to 4.5-4.7 using 10% sodium hydroxide solution (20 mL) and stirred at room temperature for 3 h at this pH. After completion of reaction pH of the reaction mixture was raised to ˜7-7.5 using 10% sodium hydroxide solution (15 mL). Separating DCM layer, aqueous layer was extracted with DCM (100 mL). The combined DCM layer was washed with water (2×50 mL). DCM layer was concentrated under vacuum at 35-40° C. completely and kept under vacuum at 35-40° C. for 1 h. The obtained solid mass was dissolved in ethyl acetate (240 mL) at 60-65° C. and cooled to 45° C. The resulting solution was filtered through 0.45 micron filter paper. To a cold (−20° C.) n-heptane (240 mL) was added the above combined ethyl acetate solution of crude linezolid at −15 to −10° C. in 25-30 min. A turbidity was appeared during addition. The suspended solution was then cooled down to −15° C. and stirred at −15 to −10° C. for 2 h. The solid mass was filtered, kept under suction for 1 h and then dried under vacuum at 50-55° C. for overnight to obtain the title compound (3.85 g) with 53% yield.

### Yield: 53%

### Polymorph: Form-I

### Enantiomer Purity: R-isomer 0.01%

### Example-23

### Preparation of (S)—N-[[3-(3-Fluoro-4-morpholinylphenyl)-2-oxo-5-oxazolidinyl]methyl acetamide, linezolid (Form-I)

To a suspension of (S)-[[N-3-(3-Fluoro-4-morpholinylphenyl)-2-oxo-5-oxazolidinyl]methyl] amine p-TSA salt (25 g, 0.0535 mol) in water (250 mL) was added 6N hydrochloric acid (25 mL) at room temperature to adjust its pH to ˜0.5. Ethyl acetate (250 mL) was added in the above solution and then readjusted its pH to 4.5-4.7 using 10% sodium hydroxide solution (60 mL). After separating ethyl acetate layer, aqueous layer was washed with ethyl acetate (2×125 mL). To the acidic aqueous layer were added DCM (250 mL) and acetic anhydride (10.92 g, 0.107 mol). The biphasic reaction mixture was adjusted its pH to 4.5-4.7 using 10% sodium hydroxide solution (50 mL) and stirred at room temperature for 3 h at this pH. After completion of reaction pH of the reaction mixture was raised to ˜7-7.5 using 10% sodium hydroxide solution (40 mL). Separating DCM layer, aqueous layer was extracted with DCM (250 mL). The combined DCM layer was washed with water (2×125 mL). DCM layer was concentrated under vacuum at 35-40° C. completely and kept under vacuum at 35-40° C. for 1 h. The obtained solid mass was dissolved in ethyl acetate (625 mL) at 60-65° C. and cooled to 50° C. The resulting solution was filtered through hyflo and washed with ethyl acetate (75 mL). To a cold (−20° C.) mixture of ethyl acetate (87.5 mL) and cyclohexane (787.5 mL) was added the above combined ethyl acetate solution of crude linezolid at −15 to −7° C. in 5-10 min and then stirred without further cooling. A turbidity was appeared during addition. The suspended solution was then cooled down to −15° C. and stirred at −15 to −10° C. for 2 h. The solid mass was filtered, kept under suction for 1 h and then dried under vacuum at 50-55° C. for overnight to obtain the title compound (10.5 g) with 58% yield.

### Yield: 58%

### Polymorph: Form-I

Polymorphic impurity: Below detection limit (slow scan count not detected)
Enantiomer Purity: R-isomer not detected

### Example-24

### Preparation of (S)—N-[[3-(3-Fluoro-4-morpholinylphenyl)-2-oxo-5-oxazolidinyl]methyl acetamide, linezolid (Form-I)

The crude linezolid (5 g) was dissolved in ethyl acetate (250 mL) at 60-65° C. and cooled to 35° C. The resulting solution was filtered through 0.45 micron filter paper. To a cold (−20° C.) n-hexane (250 mL) was added the above combined ethyl acetate solution of crude linezolid at −15 to −10° C. in 15-20 min. The turbidity was appeared during addition. The suspended solution was then cooled down to −15° C. and stirred at −10 to −5° C. for 1 h. The solid mass was filtered, kept under suction for 1 h and then dried under vacuum at 50-55° C. for 18 h to furnish the title compound (3.6 g) whose DSC and XRD matches with the standard linezolid Form-I.

### Yield: 72%

### Polymorph: Form-I

Polymorphic impurity: Below detection limit (slow scan count 629)

### Enantiomer Purity: R-isomer 0.01%

### Example-25

### Preparation of (S)—N-[[3-(3-Fluoro-4-morpholinylphenyl)-2-oxo-5-oxazolidinyl]methyl acetamide, (linezolid)

To a suspension of (R)-[[N-3-(3-Fluoro-4-morpholinylphenyl)-2-oxo-5-oxazolidinyl]methyl] amine p-TSA salt (10 g, 0.0214 mol) [obtained from (R)—[N-3-(3-Fluoro-4-morpholinylphenyl)-2-oxo-5-oxazolidinyl]methanol having S-isomer ˜0.65%] in water (100 mL) was added 6N hydrochloric acid (10 mL) at room temperature to adjust its pH to ˜0.5. Ethyl acetate (100 mL) was added in the above solution and then readjusted its pH to 4.5-4.7 using 10% sodium hydroxide solution. After separating ethyl acetate layer aqueous layer was washed with ethyl acetate (2×100 mL). To the acidic aqueous layer was added acetic anhydride (4.4 g, 0.0428 mol) at room temperature. The pH of the reaction mixture was adjusted to 4.5-4.7 using 10% sodium hydroxide solution (20 mL) and stirred at room temperature for 4 h at this pH. After completion of reaction pH of the reaction mixture was raised to ˜7-7.5 using 10% sodium hydroxide solution (20 mL). The solid was filtered from the heterogeneous reaction mass, kept under suction for 1 h and then dried under vacuum at 50-55° C. for overnight to get 3 g of the crude solid. The solid thus obtained was dissolved in a mixture (1:9) of methanol and ethyl acetate (75 mL) at 45-50° C. and recovered the solvent under vacuum at 45-50° C. The crude residue was stirred in ethyl acetate (30 mL) at room temperature for 10 min filtered and washed with ethyl acetate (2×5 mL). The solid was dried under vacuum at 45-50° C. for 12 h to furnish the title compound (2.4 g) having R-isomer 0.05%.

Polymorphic purity: Form-I
Polymorphic impurity: Below detection limit (slow scan count 629)

### Enantiomeric Purity: R-isomer 0.05%.

### Percentage Yield: 33%

### Example-26

### Preparation of (S)—N-[[3-(3-Fluoro-4-morpholinylphenyl)-2-oxo-5-oxazoli dinyl]methyl acetamide (linezolid)

To a suspension of (R)-[[N-3-(3-Fluoro-4-morpholinylphenyl)-2-oxo-5-oxazolidinyl]methyl] amine p-TSA salt (5 g, 0.0107 mol) [obtained from (R)—[N-3-(3-Fluoro-4-morpholinylphenyl)-2-oxo-5-oxazolidinyl]methanol having S-isomer ˜0.65%] in water (50 mL) was added 6N hydrochloric acid (5 mL) at room temperature to adjust its pH to ˜0.5. Ethyl acetate (50 mL) was added in the above solution and then readjusted its pH to 4.5-4.7 using 40% sodium hydroxide solution. After separating ethyl acetate layer aqueous layer was washed with ethyl acetate (2×50 mL). To the acidic aqueous layer was added acetic anhydride (2.2 g, 0.0214 mol) at room temperature. The pH of the reaction mixture was adjusted to 4.5-4.7 using 40% sodium hydroxide solution (2.5 mL) and stirred at room temperature for 4 h at this pH. After completion of reaction pH of the reaction mixture was raised to ˜7-7.5 using 40% sodium hydroxide solution (2.5 mL). The solid was filtered from the heterogeneous reaction mass, kept under suction for 1 h and then dried under vacuum at 50-55° C. for overnight to obtain the title compound (1.8 g) having R-isomer 0.05-0.06%.

Polymorphic purity: Form-II

### Enantiomeric Purity: R-isomer 0.05%.

### Percentage Yield: 50%

### Example-27

### Preparation of (S)—N-[[3-(3-Fluoro-4-morpholinylphenyl)-2-oxo-5-oxazolidinyl]methyl acetamide, (linezolid)

To a suspension of (R)-[[N-3-(3-Fluoro-4-morpholinylphenyl)-2-oxo-5-oxazolidinyl]methyl] amine p-TSA salt (5 g, 0.0107 mol) [obtained from (R)—[N-3-(3-Fluoro-4-morpholinylphenyl)-2-oxo-5-oxazolidinyl]methanol having S-isomer ˜1.9%] in water (50 mL) was added 6N hydrochloric acid (5 mL) at room temperature to adjust its pH to ˜0.5. Ethyl acetate (50 mL) was added in the above solution and then readjusted its pH to 4.5-4.7 using 40% sodium hydroxide solution. After separating ethyl acetate layer aqueous layer was washed with ethyl acetate (2×50 mL). To the acidic aqueous layer was added acetic anhydride (2.2 g, 0.0214 mol) at room temperature. The pH of the reaction mixture was adjusted to 4.5-4.7 using 40% sodium hydroxide solution (2.5 mL) and stirred at room temperature for 4 h at this pH. After completion of reaction pH of the reaction mixture was raised to ˜7-7.5 using 40% sodium hydroxide solution (2.5 mL). The solid was filtered from the heterogeneous reaction mass, kept under suction for 1 h and then dried under vacuum at 50-55° C. for overnight to obtain the title compound (2.5 g) having R-isomer 0.10%.

Polymorphic purity: Form-II

### Enantiomeric Purity: R-isomer 0.10%.

### Percentage Yield: 69%

### Example-28

### Preparation of (S)—N-[[3-(3-Fluoro-4-morpholinylphenyl)-2-oxo-5-oxazolidinyl] methyl acetamide (linezolid)

To a suspension of (R)-[[N-3-(3-Fluoro-4-morpholinylphenyl)-2-oxo-5-oxazolidinyl]methyl] amine p-TSA salt (5 g, 0.0107 mol) [obtained from (R)—[N-3-(3-Fluoro-4-morpholinylphenyl)-2-oxo-5-oxazolidinyl]methanol having S-isomer ˜1.9%] in water (50 mL) was added 6N hydrochloric acid (5 mL) at room temperature to adjust its pH to ˜0.5. Ethyl acetate (50 mL) was added in the above solution and then readjusted its pH to 4.5-4.7 using 10% sodium hydroxide solution (13 mL). After separating ethyl acetate layer aqueous layer was washed with ethyl acetate (2×50 mL). To the acidic aqueous layer was added acetic anhydride (2.2 g, 0.0214 mol) at room temperature. The pH of the reaction mixture was adjusted to 4.5-4.7 using 10% sodium hydroxide solution (7 mL) and stirred at room temperature for 4 h at this pH. After completion of reaction pH of the reaction mixture was raised to ˜7-7.5 using 40% sodium hydroxide solution (2.5 mL). The solid was filtered from the heterogeneous reaction mass, kept under suction for 1 h to get 2.9 g of crude solid. The filtrate was extracted with ethyl acetate (2×50 mL) and the combined ethyl acetate layer was washed with DM water (50 mL). The above crude solid (2.9 g) was dissolved in the ethyl acetate extract and recovered to half of its volume under vacuum at 45-50° C. The remaining clear solution was stirred at room temperature for 2 hr. The solid was filtered, washed with ethyl acetate (5 mL), then kept under suction for 30 mins and dried under vacuum at 50-55° C. for 14 h to furnish 1.8 g of the title compound having R-isomer 0.10%.

Polymorphic purity: Form-II

### Enantiomeric Purity: R-isomer 0.10%.

### Percentage Yield: 50%

### Example-29

### Preparation of (S)—N-[[3-(3-Fluoro-4-morpholinylphenyl)-2-oxo-5-oxazolidinyl]methyl acetamide (linezolid)

To a suspension of (R)-[[N-3-(3-Fluoro-4-morpholinylphenyl)-2-oxo-5-oxazolidinyl]methyl] amine p-TSA salt (5 g, 0.0107 mol) [obtained from (R)—[N-3-(3-Fluoro-4-morpholinylphenyl)-2-oxo-5-oxazolidinyl]methanol having S-isomer ˜1.9%] in water (50 mL) was added 6N hydrochloric acid (5 mL) at room temperature to adjust its pH to ˜0.5. Ethyl acetate (50 mL) was added in the above solution and then readjusted its pH to 4.5-4.7 using 10% sodium hydroxide solution (13 mL). After separating ethyl acetate layer aqueous layer was washed with ethyl acetate (2×50 mL). To the acidic aqueous layer was added acetic anhydride (2.2 g, 0.0214 mol) at room temperature. The pH of the reaction mixture was adjusted to 4.5-4.7 using 10% sodium hydroxide solution (7 mL) and stirred at room temperature for 4 h at this pH. After completion of reaction pH of the reaction mixture was raised to ˜7-7.5 using 40% sodium hydroxide solution (2.5 mL). The solid was filtered from the heterogeneous reaction mass, kept under suction for 1 h to get 2.8 g of crude solid. The solid thus obtained was suspended in a mixture of DM water (50 mL) and ethyl acetate (3 mL). The resulting mixture was stirred at 50-55° C. for 1 h and at room temperature for another 1 h. The solid was filtered, washed with DM water (2×20 mL), then kept under suction for 30 mins and dried under vacuum at 45-50° C. for 20 h to furnish 1.4 g of the title compound having R-isomer 0.07%.

Polymorphic purity: Form-II

### Enantiomeric Purity: R-isomer 0.07%.

### Percentage Yield: 39%

### Example 30

### Preparation of (S)—N-[[3-(3-Fluoro-4-morpholinylphenyl)-2-oxo-5-oxazolidinyl]methyl acetamide, linezolid (Form-I)

To a suspension of (S)-[[N-3-(3-Fluoro-4-morpholinylphenyl)-2-oxo-5-oxazolidinyl]methyl] amine pTSA salt (60 g, 0.128 mol) in water (600 mL) was added 6N hydrochloric acid (60 mL) at room temperature to adjust its pH to ˜0.5. Ethyl acetate (600 mL) was added in the above solution and then readjusted its pH to 4.5-4.7 using 10% sodium hydroxide solution (150 mL). After separating the ethyl acetate layer, aqueous layer was washed with ethyl acetate (2×300 mL). To the acidic aqueous layer were added DCM (600 mL) and acetic anhydride (26.19 g, 0.256 mol). The biphasic reaction mixture was adjusted its pH to 4.5-4.7 using 10% sodium hydroxide solution (120 mL) and stirred at room temperature for 3 h at pH 4.5-4.7. After completion of reaction, pH of the reaction mixture was raised to ˜7-7.5 using 10% sodium hydroxide solution (100 mL). Separating DCM layer, aqueous layer was extracted with DCM (600 mL). The combined DCM layer was washed with water (2×300 mL). DCM layer was concentrated completely under atmospheric pressure at 40-45° C. and kept under vacuum at 40-45° C. for 1 h. The obtained solid mass was dissolved in ethyl acetate (1080 mL) at 70-75° C. To a cold (−10° C.) ethyl acetate (120 mL) was added the above hot (70-75° C.) ethyl acetate solution of crude linezolid at −10 to +15° C. in 20-30 min and stirred at ambient temperature for 5-10 min to stabilize the mass temperature without external cooling. The suspended solution was then cooled down to −5° C. and stirred at 0 to −5° C. for 1 h. The solid mass was filtered, kept under suction for 1 h and then dried under vacuum at 100-105° C. for overnight to obtain the title compound as Polymorphic Form-I. (Yield: 29 gm, 67%; HPLC Purity: >99.8%; Polymorphic impurity: Below detection limit)

### Example 31

### Isolation of Crude Linezolid from Mother Liquor

Filtrate (1200 mL) of the above whole batch (60 gm of (S)—N-[[3-(3-Fluoro-4-morpholinylphenyl)-2-oxo-5-oxazolidinyl]methyl]amine p-TSA salt) was concentrated under vacuum at 45-50° C. to a volume of 200-220 mL. The resulting solution was heated to 78° C. and stirred 75-78° C. for 30 mins. The solution was slowly cooled to 30° C. with occasional stirring and then further cooled to −12° C. under stirring. The solid was precipitated out and kept stirring at −10 to −12° C. for 2 h. The solid was filtered and dried at 100-105° C. for 24 h to get crude linezolid (Form-I). (Wet weight: 5.7-6.2 gm; Dry weight: 5.5-6.0 gm; HPLC Purity: >99.5%)

### Example 32

### Preparation of Form-I of Linezolid by Agitated Thin Film Drying (ATFD)

To a stirred mixture of methanol (20 mL) and ethyl acetate (80 mL) was added linezolid (5.0 g) at 25-30° C. The mixture was warmed to 45-50° C. and stirred at this temperature for 15-20 minutes to get a clear solution. The resulting solution was fed into Rotavapor at temperature: 65-75° C. After completion of feeding the mass was kept under vacuum at 70-75° C. The solid was further dried under vacuum at 100-105° C. for 48 hours to provide Form-I of linezolid.

### Example 33

### Preparation of Form-I of Linezolid by Agitated Thin Film Drying (ATFD)

To a stirred mixture of methanol (15 mL) and ethyl acetate (135 mL) was added linezolid (5.0 g) at 25-30° C. The mixture was warmed to 45-50° C. and stirred at this temperature for 15-20 minutes to get a clear solution. The resulting warmed (40-45° C.) solution was fed into Rotavapor at temperature: 80-95° C. After completion of feeding the mass was kept under vacuum at 90-95° C. for 1 hour. The solid was further dried under vacuum at 100-105° C. for 18 hours to provide Form-I of linezolid.

### Example-34

### Preparation of (S)—N-[[3-(3-Fluoro-4-morpholinylphenyl)-2-oxo-5-oxazolidinyl]methyl acetamide, linezolid (Form-I)

To a suspension of (S)-[[N-3-(3-Fluoro-4-morpholinylphenyl)-2-oxo-5-oxazolidinyl]methyl] amine pTSA salt (60 g, 0.128 mol) in water (600 mL) was added 6N hydrochloric acid (60 mL) at room temperature to adjust its pH below 1. Ethyl acetate (600 mL) was added in the above solution and then readjusted its pH to 4.5-4.7 using 10% sodium hydroxide solution (150 mL). After separating ethyl acetate layer aqueous layer was washed with ethyl acetate (2×300 mL). To the acidic aqueous layer were added DCM (600 mL) and acetic anhydride (26.19 g, 0.256 mol). The biphasic reaction mixture was adjusted its pH to 4.5-4.7 using 10% sodium hydroxide solution (120 mL) and stirred at room temperature for 3 h at this pH. After completion of reaction pH of the reaction mixture was raised to ˜7-7.5 using 10% sodium hydroxide solution (˜100 mL). Separating DCM layer, aqueous layer was extracted with DCM (600 mL). The combined DCM layer was washed with water (2×300 mL). After treating with activated carbon DCM layer was concentrated under vacuum at 35-40° C. completely and kept under vacuum at 35-40° C. for 1 h. The obtained solid mass was dissolved in ethyl acetate (1200 mL) at 75-78° C. and cooled to 70° C. The resulting solution was filtered through hyflo bed. To a cold (−10° C.) mixture of cyclohexane (270 mL) and ethyl acetate (30 mL) was added the above combined ethyl acetate solution of crude linezolid at −10 to +10° C. over 20-30 min and stirred for 10 min without further cooling. The suspended solution was then cooled down to −5° C. and stirred at −5 to −8° C. for 2 h. The solid mass was filtered, kept under suction for 30 min and then dried under vacuum at 85-90° C. for overnight to obtain the title compound (28.5 g) with 66.4% yield.

### Yield: 66.4%

### Polymorph: Form-I

Polymorphic impurity: Below detection limit

What is claimed is:

1. A process of preparation of enantiomerically pure Linezolid Form-I comprising converting a substantially enantiomerically pure linezolid hydroxide compound of formula II to Linezolid Form I compound of formula I.

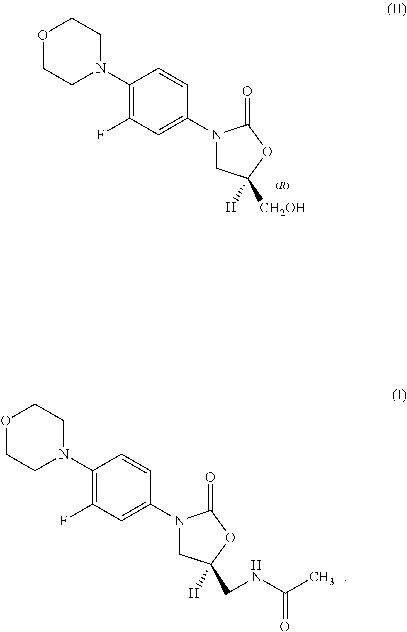

2. The process of claim 1, wherein converting the substantially enantiomerically pure linezolid hydroxide compound of formula II to the Linezolid Form I compound of formula I comprises acetylation of (S)—N-[[3-(3-fluoro-4-morpholinylphenyl)-2-oxo-5-oxazolidinyl]methyl]amine or salt thereof, with a proviso that acetylation reaction is not carried out in presence of a ketonic solvent.

3. The process of claim 1, wherein said linezolid Form I is substantially free of residual solvent(s), with S-isomer content more than about 99.95% relative to R-isomer.

4. The process of claim 3, wherein said residual solvent(s) is less than about 1200 ppm.

5. The process of claim 3, wherein said residual solvent(s) is less than about 1000 ppm.

6. The process of claim 1, wherein the enantiomerically pure Linezolid hydroxide is prepared by a process comprising the steps of: 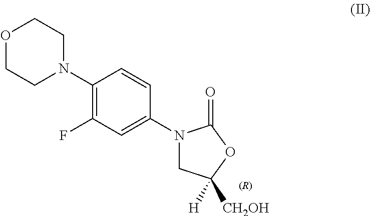
a. contacting linezolid hydroxide and a solvent to form a solution;
b. optionally adjusting the moisture content of the solution of step (a) to between 0.2 and 0.6% w/w;
c. optionally adding an anti-solvent; and
d. isolating enantiomerically pure linezolid hydroxide compound of formula II.

7. The process of claim 6, wherein the solvent is selected from the group consisting of methyl acetate, ethyl acetate, n-propyl acetate, isopropyl acetate and n-butyl acetate.

8. The process of claim 6, wherein the antisolvent is selected from the group consisting of pentane, hexane, cyclohexane, heptane, octane, methylcyclohexane, chloronaphthalene, orthodichlorbenzene, toluene, ethylbenzene, isopropylbenzene and diethylbenzene.

9. The process of claim 1, further comprising the steps of:
a. providing a solution or a slurry or a suspension of linezolid in a solvent selected from the group consisting of ester solvents, halogenated solvents, ketonic solvents, and ethers solvents at a temperature of from about 30° C. to about 150° C.;
b. mixing a solvent or optionally an antisolvent with the solution or slurry or suspension as obtained from step (a) at temperature lower than the-temperature of step (a); and
c. isolating enantiomerically pure Linezolid Form-I.

10. The process of claim 9, wherein the antisolvent is a hydrocarbon.

11. The process of claim 9, wherein the Linezolid Form-I is further converted into any other polymorphic form of Linezolid.

12. The process of claim 9, wherein said process produces stable crystalline form of Linezolid Form I.

13. The process of claim 12, comprising the steps of:
(a) providing a solution of linezolid in a solvent at a first temperature, wherein the first temperature is between about 55° C. and a refluxing temperature of the solvent system;
(b) adding the solution obtained in step (a) into a pre-cooled solvent at a second temperature, wherein the temperature of the pre-cooled solvent is from about −10° C. to about −5° and the second temperature is a temperature ranging from about −10° C. to about 20° C.;
(c) stirring the solution of step (b) at a temperature which is not more than about 5° C.;
(d) optionally repeating the steps (b) and (c);
(e) isolation of substantially solvent-free crystals of the Linezolid Form-I; and
(f) drying the material obtained in step (e) at a temperature above about 90° C., wherein the solvent used in steps (a) and (b) is independently selected from the group consisting of an ester, an alcohol, a nitrile, a ketone, an ether, an amide, a dialkylsulfoxide solvent, a chlorinated solvent and a mixture thereof.

14. The process of claim 12, comprising the steps of:
a. providing a solution of linezolid in an organic solvent or mixture thereof or a mixture of organic solvent and water, wherein the organic solvent used in steps (a) is selected from the group consisting of esters, alcohols, nitriles, ketones, ethers, amides, dialkylsulfoxide, chlorinated solvents and mixtures thereof;
b. removing solvent using agitated thin film drying;
c. drying at about 90-120° C.; and
d. isolating the Linezolid Form I.

15. The process according to the claim 14, wherein said Linezolid of Form I is obtained by washing the solution of (S)-[[N-3-(3-Fluoro-4-morpholinylphenyl)-2-oxo-5 oxazolidinyl]methyl] amine p-TSA salt in hydrochloric acid at pH to 4.5-4.7 with ester solvent, followed by treatment with acetic anhydride.

16. The process according to the claim 14, wherein said Linezolid of Form I is obtained by extraction of the reaction mixture of (S)-[[N-3-(3-Fluoro-4-morpholinylphenyl)-2-oxo-5-oxazolidinyl]methyl] amine p-TSA salt, acetic anhydride and water at pH to 7-7.5 using chlorinated solvent.

17. A substantially enantiomerically pure linezolid hydroxide compound of formula II having R-isomer content more than 99.9% relative to its S-isomer 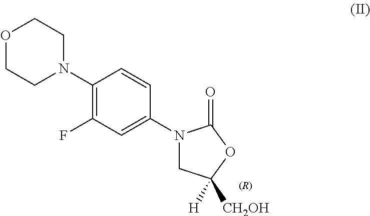
wherein said substantially enantiomerically pure linezolid hydroxide is prepared by the process of claim 6.
